(19) United States
(12) Patent Application Publication
(10) Pub. No.: US 2021/0293773 A1
(43) Pub. Date: Sep. 23, 2021

(54) FUEL EVALUATION APPARATUS, CONTROL METHOD THEREFOR, AND COMPUTER-READABLE STORAGE MEDIUM

(71) Applicant: HONDA MOTOR CO., LTD., Tokyo (JP)

(72) Inventors: Tomohide Haraguchi, Wako-shi (JP); Takahiro Gunji, Wako-shi (JP)

(73) Assignee: HONDA MOTOR CO., LTD., Tokyo (JP)

(21) Appl. No.: 17/340,705

(22) Filed: Jun. 7, 2021

Related U.S. Application Data
(63) Continuation of application No. PCT/JP2018/045718, filed on Dec. 12, 2018.

(51) Int. Cl. G01N 33/22; G07C 5/08; G06Q 30/02

(52) U.S. Cl. CPC G01N 33/22; G07C 5/0808; G06Q 30/0206; G06Q 30/0201; G06Q 30/0215

(57) ABSTRACT

There is provided a fuel evaluation apparatus for evaluating fuel supplied to a vehicle. The fuel evaluation apparatus performs: acquiring information on a remaining capacity of a fuel storage unit in the vehicle; acquiring information on an alcohol concentration of fuel within the fuel storage unit; calculating a supply amount of fuel supplied to the fuel storage unit, based on the acquired information on the remaining capacity; evaluating the alcohol concentration of the supplied fuel, based on the calculated supply amount of fuel and a change in the alcohol concentration before and after supply of the fuel to the fuel storage unit that is based on the acquired information on the alcohol concentration; and transmitting fuel information comprising the calculated supply amount of fuel and a result of the evaluation to external apparatus determining incentives according to the fuel information.

### CROSS-REFERENCE TO RELATED APPLICATION(S)

This application is a continuation of International Patent Application No. PCT/JP2018/045718 filed on Dec. 12, 2018, the entire disclosure of which is incorporated herein by reference.

### BACKGROUND OF THE INVENTION

### Field of the Invention

The present invention relates to a fuel evaluation apparatus, a control method therefor, and a computer-readable storage medium.

### Description of the Related Art

Conventionally, fossil fuels such as gasoline are used as fuel for cars, whereas usage of renewable energy that is typified by carbon offset alternative fuels such as biofuels and e-fuels is recommended from the viewpoint of countering global warming and the like.

For example, Japanese Patent Laid-Open No. 2004-152138 proposes a technique for virtually achieving zero CO2 emissions when fuels sold to customers are used, by calculating the amount of CO2 emissions accompanying use of fuels that have been sold, and offsetting emission credits equivalent to the amount of CO2 emissions with CO2 emission credits that the seller of the fuel has acquired in advance.

Also, Japanese Patent Laid-Open No. 2007-287112 proposes a method for utilizing membership point cards to notify users of the amount of CO2 reduction, with the aim of propagating the sale of 100% bioethanol automotive fuel.

### SUMMARY OF THE INVENTION

Policies and legislation such as incentives being provided to producers and importers of alternative fuels with low carbon intensity are being advanced nationally. However, the current situation is that how incentives are acquired and utilized is left up to the individual business operators, which is limiting as a way of leading to expanded usage of alternative fuels by general users.

In view of this, an object of the present invention is to improve the convenience with which general users acquire and use incentives, with the aim of further expanding the usage of alternative fuels.

In order to solve the above problem, the instant invention has the following configuration. That is, a fuel evaluation apparatus for evaluating fuel supplied to a vehicle, the fuel evaluation apparatus performing a method comprising: acquiring information on a remaining capacity of a fuel storage unit provided in the vehicle; acquiring information on an alcohol concentration of fuel within the fuel storage unit; calculating a supply amount of fuel supplied to the fuel storage unit, based on the acquired information on the remaining capacity; evaluating the alcohol concentration of the fuel supplied to the fuel storage unit, based on the calculated supply amount of fuel and a change in the alcohol concentration before and after supply of the fuel to the fuel storage unit that is based on the acquired information on the alcohol concentration; and transmitting fuel information comprising the calculated supply amount of fuel and a result of the evaluation to external apparatus determining incentives according to the fuel information.

### BRIEF DESCRIPTION OF THE DRAWINGS

FIG. 1 is a diagram showing an outline of the overall system according to the present invention.

FIG. 2A is a diagram showing an example of the hardware configuration of respective apparatuses according to the present invention.

FIG. 2B is a diagram showing an example of the hardware configuration of respective apparatuses according to the present invention.

FIG. 3A is a diagram showing an example of the software configuration of respective apparatuses according to the present invention.

FIG. 3B is a diagram showing an example of the software configuration of respective apparatuses according to the present invention.

FIG. 4 is a diagram for illustrating the data flow according to the present invention.

FIG. 5 is a flowchart of fuel evaluation processing according to a first embodiment.

FIG. 6 is a flowchart of incentive provision processing according to the present invention.

FIG. 7 is a flowchart of fuel evaluation processing according to a second embodiment.

### DESCRIPTION OF EMBODIMENTS

Hereinafter, embodiments according to the present invention will be described using the drawings. Note that the configurations and the like shown below are given as examples, and the present invention is not limited thereto.

### Credits

Businesses and the like are working to keep emissions within given levels, with the aim of reducing greenhouse gases. On the other hand, in the case where the targeted amount of reduction cannot be achieved, the part that cannot be achieved is offset (carbon offset) by purchasing credits.

For example, fossil fuels are used as fuel for cars and the like. In contrast, use of alternative fuels such as biofuels is recommended due to the ecological focus of recent years. As one of these policies, there are cases where it is mandatory for fuels to contain a certain renewable fuel (e.g., ethanol) as a standard. When a vendor that manufactures and sells fuel buys in a renewable fuel that is used in order to manufacture fuel, credits (e.g., carbon credits) that are based on the reduction in greenhouse gases achieved by the manufactured fuel being consumed as a substitute for fossil fuels are issued together therewith. Issued credits can be used in offsetting emissions as described above, and can be traded between vendors separately from renewable fuels. The market price for credits fluctuates according to supply and demand between vendors. Such credits that can be bought and sold include Renewable Identification Numbers (RINs) under the Renewable Fuel Standard (RFS) in the U.S., and California's low carbon fuel standard (LCFS) administered by the California Air Resources Board (CARB), for example.

The description of the following embodiments according to the present invention also takes trading of credits such as described above into consideration.

### First Embodiment

### System Outline

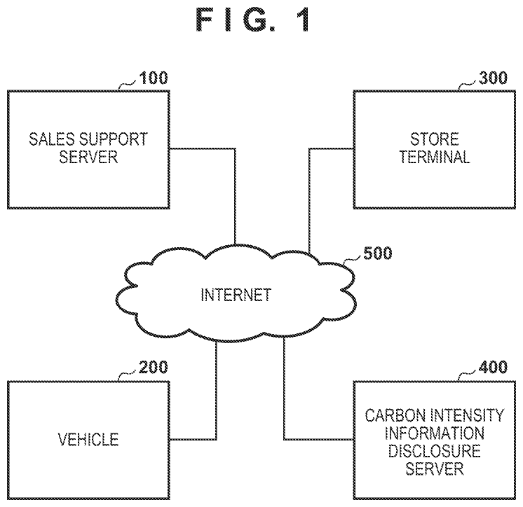

FIG. 1 is a diagram for illustrating an outline of the overall system according to one embodiment of the present invention. In the system according to the present embodiment, a sales support server 100, a vehicle 200, a store terminal 300 and a carbon intensity information disclosure server 400 are connected in a communicable manner via an internet 500. The sales support server 100 collects and manages information such as on stores that undertake selling and the like of alternative fuels and on users who undertake buying and the like of alternative fuels, and performs processing relating to sales promotion of alternative fuels. The vehicle 200 is a vehicle that can be driven using an alternative fuel, such as a four-wheeled vehicle and a two-wheeled vehicle, for example. The store terminal 300 is an information processing apparatus that is used at a store that undertakes buying in, selling and the like of alternative fuels, with a PC (Personal Computer) and a dedicated terminal being given as examples, but is not particularly limited thereto. Note that the store terminal 300 does not necessarily need to be where the store is located. The carbon intensity information disclosure server 400 that functions as a fuel information disclosure server discloses, as fuel information, carbon intensity information on fuels containing alternative fuels. Specifically, the emission amount of carbon dioxide that is produced is shown relative to the amount of fuel that is consumed. The carbon intensity information disclosure server 400 is provided by a public institution, for example, and disclosure information thereof is secured.

In FIG. 1, the apparatuses shown above are shown individually, but more apparatuses may be provided, according to factors such as the number of stores, the number of users, and distribution of the processing load.

### Example Hardware Configuration

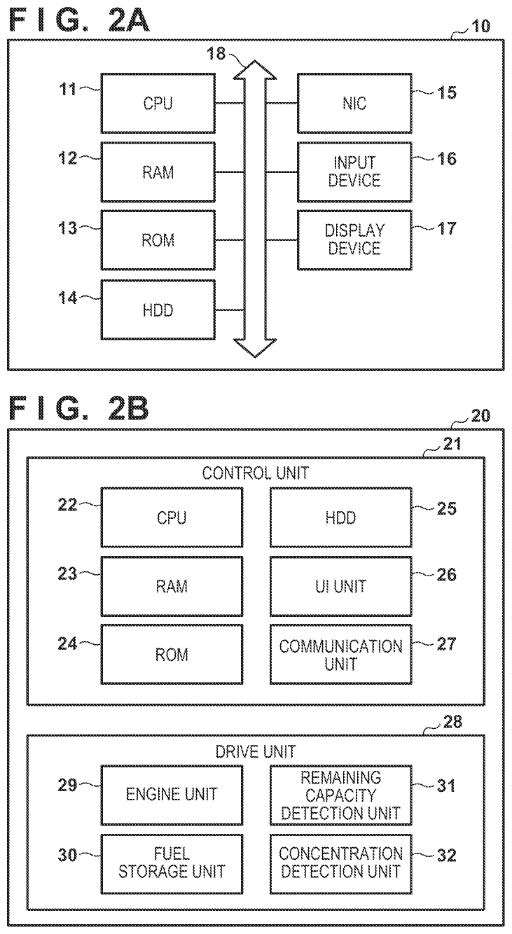

An example configuration of the hardware of respective apparatuses according to the present embodiment will be described using FIGS. 2A and 2B.

### Information Processing Apparatus

FIG. 2A is a diagram showing an example of the hardware configuration of an information processing apparatus according to the present embodiment. Here, description will be given taking the sales support server 100 and an information processing apparatus 10 that is operable as the store terminal 300 as an example. The apparatuses are described here as all being provided with the same configuration, but may be respectively provided with different configurations.

The information processing apparatus 10 is constituted to include a CPU 11, a RAM 12, a ROM 13, a HDD 14, a display device 17, an input device 16, and a NIC 15. The respective parts are connected in a communicable manner to each other via an internal bus 18. The CPU 11 administers overall control of the information processing apparatus 10. The RAM (Random Access Memory) 12 is a volatile storage area and is used as a work memory and the like. The ROM (Read Only Memory) 13 is a nonvolatile storage area, and various programs and data are held thereon. The HDD (Hard Disk Drive) 14 is a nonvolatile storage area, and various data is held thereon.

The display device 17 displays various data to the user. The input device 16 receives inputs and operations from the user. The NIC (Network Interface Card) 15 administers communication with external devices via the internet 500, and transmits and receives various data. The communication method referred to here may be wired or wireless and is not limited in terms of the communication system, and communication may be performed using a combination of communication systems.

### Vehicle

FIG. 2B shows an example of the hardware configuration of a vehicle according to the present embodiment. Note that only portions required in describing the present embodiment will be illustrated here, and detailed description of parts related to travel and the like will be omitted.

The vehicle 200 is constituted to include a control unit 21 and a drive unit 28. The control unit 21 is constituted to include a CPU 22, a RAM 23, a ROM 24, an HDD 25, a UI unit 26, and a communication unit 27. The respective parts are connected in a communicable manner to each other via an internal bus (not shown). The CPU 22 administers overall control of the control unit 21. The RAM (Random Access Memory) 23 is a volatile storage area, and is used as a work memory and the like. The ROM (Read Only Memory) 24 is a nonvolatile storage area, and various programs and data are held thereon. The HDD (Hard Disk Drive) 25 is a nonvolatile storage area, and various data is held thereon.

The UI unit 26 is a user interface with the user and performs processing such as receiving operations from the user and displaying information. The communication unit 27 administers communication with external devices via the internet 500, and transmits and receives various data. The communication method referred to here is not limited in terms of the communication system, and communication may be performed using a combination of communication systems.

The drive unit 28 is a part related to travel of the vehicle 200, and is constituted to include an engine unit 29, a fuel storage unit 30, a remaining capacity detection unit 31, and a concentration detection unit 32. The engine unit 29 is a part for causing the vehicle 200 to travel using fuel stored in the fuel storage unit 30. The fuel storage unit 30 is a part (fuel tank, etc.) that stores fuel for causing the vehicle 200 to travel, and, in the present embodiment, is capable of storing fuel containing an alternative fuel as a component. The remaining capacity detection unit 31 detects the remaining capacity of fuel that is stored in the fuel storage unit 30. The remaining capacity referred to here may be detected using a level gauge that detects the remaining amount in a stepwise manner, or a configuration capable of finer detection may be adopted. The concentration detection unit 32 is a part that detects the concentration of a predetermined component contained in the fuel that is stored in the fuel storage unit 30. In the present embodiment, description is given taking the concentration of ethanol, which is an alternative fuel, as the predetermined component. Also, in the present embodiment, an example will be described in which an alcohol sensor capable of detecting the concentration of ethanol is used as the concentration detection unit 32.

### Example Software Configurations

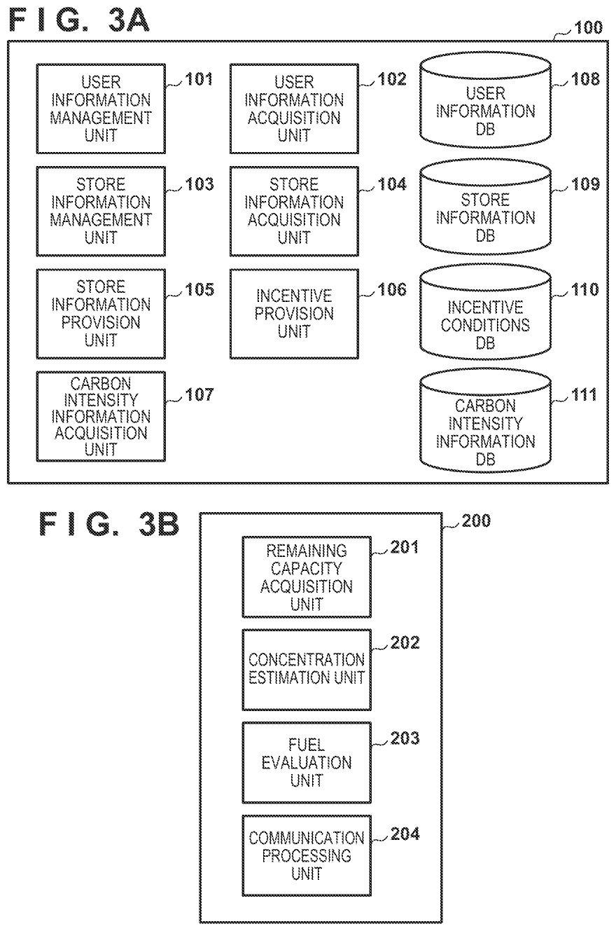

Example configurations of the software of an information processing apparatus that functions as the sales support server 100 according to the present embodiment and the vehicle 200 will be described, using FIGS. 3A and 3B.

FIG. 3A shows an example configuration of the software of the sales support server 100 according to the present embodiment. In the present embodiment, the respective processing units are realized by programs stored in a storage unit such as the HDD 14 being read out and executed by the CPU 11 provided in the information processing apparatus 10. Databases (DB) may be constituted in different storage units or may be constituted in one storage unit.

The sales support server 100 according to the present embodiment is constituted to include a user information management unit 101, a user information acquisition unit 102, a store information management unit 103, a store information acquisition unit 104, a store information provision unit 105, an incentive provision unit 106, and a carbon intensity information acquisition unit 107. Furthermore, the sales support server 100 is constituted to include a user information DB 108, a store information DB 109, an incentive conditions DB 110, and a carbon intensity information DB 111.

The user information management unit 101 manages the various user information held in the user information DB 108. Also, the user information management unit 101 performs registration and updating of user information, in response to requests from the user. The user information acquisition unit 102 acquires user information from the user via a user terminal such as a PC, and registers the user information in the user information DB 108. For example, the user information acquisition unit 102 may provide a UI (User Interface) or the like for when the user registers user information as a web page.

The store information management unit 103 manages the various store information held the store information DB 109. The store information acquisition unit 104 acquires store information from the store side via the store terminal 300, and registers the store information in the store information DB 109. For example, the store information acquisition unit 104 may provide a UI or the like for when registering store information on the store side as a web page. The store information provision unit 105 provides store information that is managed in the store information DB 109 and incentive conditions that are managed in the incentive conditions DB 110, in response to requests from the user and the like. As for the store information provided here, provision of position information of nearby stores based on the position of the user (vehicle 200) and information on fuel products carried by the stores are given as an example. Also, as for the incentive conditions provided here, information on incentives that are obtained when fuel products are purchased at corresponding stores is given as an example. Incentive conditions may include conditions determined for every store and conditions determined for every fuel product carried by the store.

The incentive provision unit 106 provides incentives to the user, based on incentive conditions held in the incentive conditions DB 110, the purchase history of fuel products by the user, and the like. The incentives provided here are managed in association with users in the user information DB 108. Incentive conditions are, for example, conditions for determining incentives to be provided that are defined according to the amount of emissions of CO2 (carbon dioxide) that is produced according to the alternative fuel component (e.g., alcoholic mixture concentration) constituting a fuel product, the amount of CO2 reduction from a reference value according to usage of the fuel product, and the like. As for the reference value referred to here, the amount of CO2 produced per unit that is prescribed relative to the case where a fossil fuel is consumed is given as an example. Accordingly, a relative value that is reduced in the case where an alternative fuel is used relative to this reference value may be used as for the amount of CO2 reduction. Specifically, the incentive per unit amount of the purchased fuel product or the like is used. Information on incentive conditions may be provided to users who it is envisaged will purchase the fuel product. The incentive conditions may, for example, be determined based on trading information of credits that are issued and traded following the distribution of renewable fuels (e.g., ethanol) that are contained as a component in fuel products. Credits referred to here include credits that can be bought and sold in market trading by vendors, with Renewable Identification Numbers (RINs) as described above being given as an example thereof. Note that trading information may be acquired from the store terminal 300 or from an external server (not shown) as publically released market price information. The carbon intensity information acquisition unit 107 acquires carbon intensity information from the carbon intensity information disclosure server 400. The carbon intensity information acquired here is held in the carbon intensity information DB 111. As described above, the amount of emissions of CO2 produced relative to the amount of alternative fuel consumed, for example, is shown as carbon intensity information.

Note that the respective means of the sales support server 100 shown in FIG. 3A may be constituted by load sharing with a plurality of information processing apparatuses. Also, the respective databases are not limited to being held by the server that has the respective means, and may be constituted in a device connected in a communicable manner via a network. In this case, it becomes possible for the server to acquire required information, by accessing the external device and referring to the various databases. Also, a configuration may be adopted in which some of the means shown in FIG. 3A are realized on the terminal device side (or by an application in the terminal device).

FIG. 3B shows an example configuration of the software of the vehicle 200 according to the present embodiment. In the present embodiment, the respective processing units are realized by programs stored in a storage unit such as the HDD 25 being read out and executed by the CPU 22 provided in the vehicle 200.

The vehicle 200 is constituted to include a remaining capacity acquisition unit 201, a concentration estimation unit 202, a fuel evaluation unit 203, and a communication processing unit 204. The remaining capacity acquisition unit 201 acquires information on the remaining capacity of fuel in the fuel storage unit 30, based on the result detected by the remaining capacity detection unit 31 of the vehicle 200. The remaining capacity referred to here is assumed to be acquired at a predetermined time interval, at the timing at which fuel is supplied, and the like. Note that the information on the remaining capacity is assumed to be held in the HDD 25 or the like as history data. The concentration estimation unit 202 estimates the concentration of a predetermined component in the fuel that is stored in the fuel storage unit 30. The predetermined component referred to here means ethanol or the like serving as an alternative fuel. The concentration estimation unit 202 estimates the concentration, based on the variation in the remaining capacity acquired by the remaining capacity acquisition unit 201 and the result detected by the concentration detection unit 32. The fuel evaluation unit 203 evaluates fuel supplied to the fuel storage unit 30, based on the volume of supplied fuel acquired by the remaining capacity acquisition unit 201 and the concentration of fuel estimated by the concentration estimation unit 202. The communication processing unit 204 performs processing for transmitting information on the fuel evaluated by the fuel evaluation unit 203 to the sales support server 100 using the communication unit 27. Also, the communication processing unit 204 receives store information provided by the sales support server 100, information on provided incentives, and the like.

### Data Flow

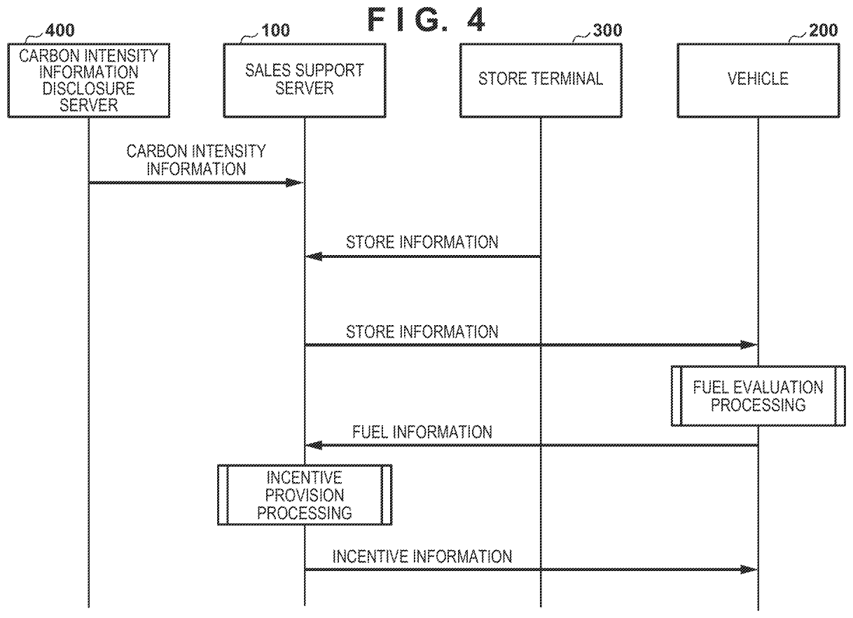

The flow of various data between the apparatuses according to the present embodiment will be described using FIG. 4. Note that, in FIG. 4, the data transmission/reception timing is not strictly prescribed, and the data transmission/reception timing and frequency may vary and shift, according to the timing at which requests and the like arise. Also, user information of the user of the vehicle 200 is assumed to be registered in advance and managed by the sales support server 100, based on user operations using an information processing apparatus such as a PC. As for the user information referred to here, a user account, authentication information (password, etc.), name, age, sex, place of residential, information on the vehicle owned, and contact details (mail address, etc.) are given as examples. Also, position information of the vehicle 200 that is used by the user may be periodically acquired, apart from the position (address) serving as a base.

The sales support server 100 acquires carbon intensity information from the carbon intensity information disclosure server 400. The acquisition referred to here may be periodical acquisition or acquisition at a timing defined in advance. As described above, the carbon intensity information referred to here includes information on the carbon intensity of alternative fuels. Since alternative fuels differs in terms of the manufacturing process and materials depending on the manufacturer and the like, carbon intensity information for each alternative fuel is desirably acquired.

Next, data communication between the sales support server 100 and the store terminal 300 will be described. The store terminal 300 transmits its own store information to the sales support server 100. As for the store information referred to here, position information of the store, business hours, prices (e.g., unit price) of fuel products carried by the store, product characteristics (e.g., type of fossil fuels, renewable fuels, etc.), quality (e.g., alcoholic mixture concentration) and promotional information of the store are given as examples. Also, the store terminal 300 transmits trading information relating to buying in of alternative fuels purchased by the vendor to which the store terminal 300 belongs to the sales support server 100. The trading information referred to here includes the amount of credits that the vendor has, their market price, and the like. Note that, here, an example in which trading information is provided by the store terminal 300 is shown, but the present invention is not limited thereto, and, for example, a configuration may be adopted in which a vendor running a plurality of stores collectively provides trading information from a different terminal.

The sales support server 100 provides store information to the vehicle 200, based on store information, user information and the like managed thereby. The store information referred to here includes information on stores near the position of the user (vehicle 200), information on products carried by the stores, promotional information of the stores, and incentive conditions of the stores. Product information may include information on the effect (amount of CO2 reduction, etc.) on the environment of purchasing and using respective products. The provision timing may be triggered by a request from the vehicle 200 or a configuration may be adopted in which store information is presented every preset time or interval.

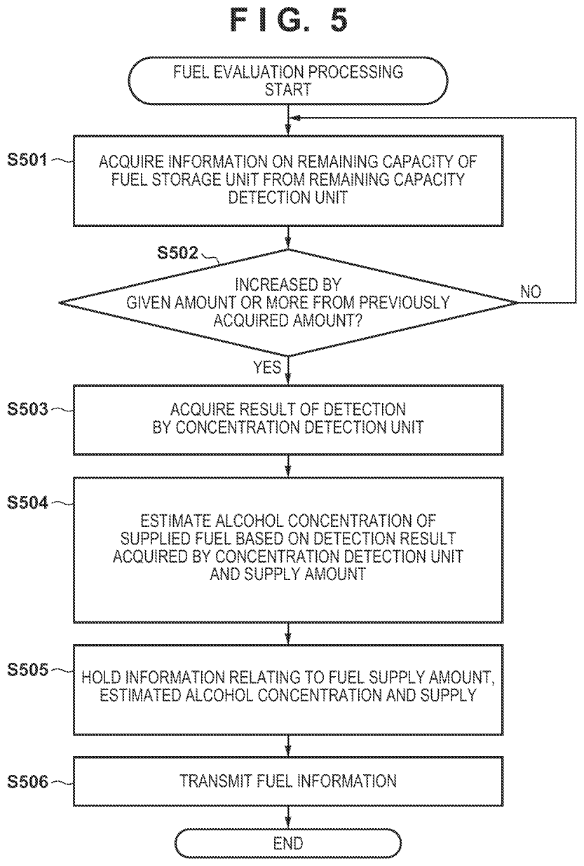

When the user supplies fuel to the vehicle 200, the vehicle 200 perform processing for evaluating the fuel, and transmits information thereof (hereinafter, referred to as “fuel information”) to the sales support server 100. Fuel evaluation processing will be described using FIG. 5.

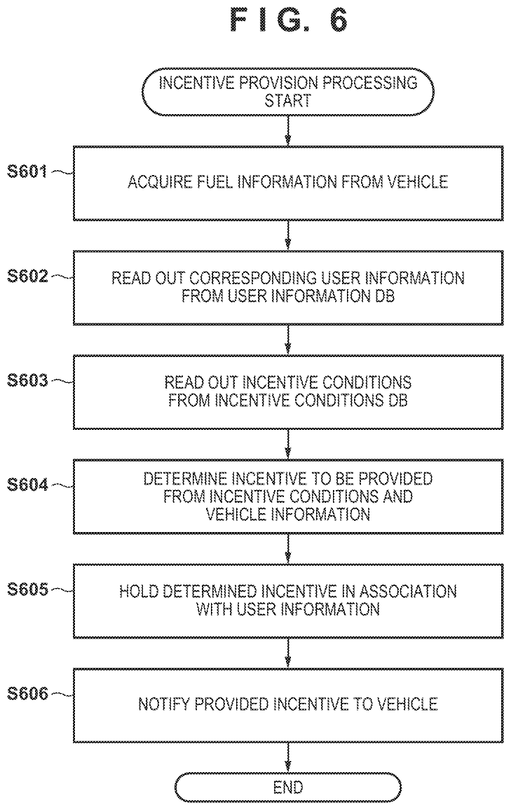

The sales support server 100, in response to having received fuel information from the vehicle 200, performs incentive provision processing for providing an incentive that is based on the fuel information. Incentive provision processing will be described later using FIG. 6. Thereafter, information relating to the provided incentive is notified to the vehicle 200. The notification referred to here may be notified every predetermined interval, or may be notified as a response to the timing at which purchase information is received.

### Provision of Store Information to Vehicle

The sales support server 100 according to the present embodiment provides the store information of stores that sell fuel products to the vehicle 200. As an example of the provision method referred to here, store information may, for example, be provided to a car navigation system (not shown) provided in the vehicle 200. A configuration may be adopted in which information on a nearby store is provided, according to position information obtained by the GPS (Global Positioning System) function of the car navigation system. Product information on fuel products associated with the store and information (incentive conditions) on incentives that are obtained through purchase can also be collectively provided to the user.

Also, in addition to store information, the sales support server 100 may provide various advertising associated with products or the store via an application. Also, the route to the store, congestion conditions in the store and the like may also be collectively provided.

### Incentives

Incentives according to the present embodiment will be described. In the present embodiment, an incentive is provided to the user, according to the evaluation result of a fuel containing an alternative fuel that is supplied to the vehicle 200. The incentives referred to here include, for example, points that are provided according to the amount of CO2 reduction, expense rewards (cash back) that are calculated from the relationship between the amount of alternative fuel contained in the fuel and the trading price of credits, and discount services. Also, discount services may be services that can be used when purchasing or receiving provision of products (e.g., vehicles, services) that are eco-oriented, apart from fuel products containing alternative fuel. Also, incentives need not be limited to those specific to the vendor to which the store that sells the fuel products belongs, and may, for example, be incentives that are can be used between vendors.

### Processing Flow

### Fuel Evaluation Processing

The flow of processing for evaluating supplied fuel in the vehicle 200 will be described using FIG. 5. This processing is realized as a result of the various processing units shown in FIG. 3B being realized by programs held in the HDD 25 or the like being read out and executed by the CPU 22 of the vehicle 200. This processing may, for example, be started when the ignition of the vehicle 200 is switched ON/OFF, or may be started every predetermined time. Alternatively, this processing may be executed according to a specific event such as when the vehicle 200 is stationary. Note the present invention is not limited thereto, and this processing may be executed at other timings.

In step S501, the remaining capacity acquisition unit 201 acquires information on the remaining capacity of fuel that is stored in the fuel storage unit 30 from the remaining capacity detection unit 31. The information acquired here is stored in a storage unit such as the HDD 25 as history information according to changes therein.

In step S502, the remaining capacity acquisition unit 201 determines whether the remaining capacity has increased by a given amount or more as the amount of change from the previous remaining capacity, with reference to the history of acquired information on the remaining capacity. The given amount referred to here is assumed to be an amount defined in advance and held in the ROM 24 or the like. In the case of detecting the remaining capacity with a level gauge, for example, the remaining capacity may be determined to have increased, if the level has increased by one or more. If it is determined that the amount of change is greater than or equal to the given amount (YES in step S502), the processing advances to step S503 assuming that fuel has been newly supplied, and, if it is determined that the amount of change has decreased or has increased by less than the given amount (NO in step S502), the processing returns to step S501, and acquisition of information on the remaining capacity is continued.

In step S503, the concentration estimation unit 202 acquires information relating to the concentration of a predetermined component contained in the fuel that is stored in the fuel storage unit 30 from the concentration detection unit 32. As described above, in the present embodiment, an alcohol sensor is used as the concentration detection unit 32, and the concentration referred to here is assumed to be the alcohol concentration. Note that information on the alcohol concentration detected by the concentration detection unit 32 is stored in a storage unit such as the HDD 25 as history information according to changes therein. Also, a configuration may be adopted in which the detection of the alcohol concentration by the concentration detection unit 32 referred to here is continued until the vehicle 200 has traveled a given distance or traveled for a given time after a given amount of fuel is supplied. Alternatively, detection may be continued until the detection results by the concentration detection unit 32 have stabilized to a certain extent. The given distance and given time referred to here are assumed to be defined in advance. The components of the fuel in the fuel storage unit 30 are agitated through vibration and the like and become uniformly dispersed, thereby enabling the accuracy with which supplied fuel is detected to be improved.

In step S504, the concentration estimation unit 202 estimates the alcohol concentration of the supplied fuel, based on the remaining capacity of fuel before supply, the supply amount of fuel, the alcohol concentration before supply, and the alcohol concentration after fuel supply. Note that, in the case where a given distance or a given time is provided in order to detect the alcohol concentration, the alcohol concentration may be estimated with consideration for the change in fuel consumed over that distance or time.

In step S505, the fuel evaluation unit 203 stores the supplied amount of fuel, estimated alcohol concentration, supply date-time, supply place (position) and the like in association with each other in a storage unit such as the HDD 25 as information on the supply history. At this time, a configuration may be adopted in which the previous supply information is referred to and eco-related evaluation is performed from the result of comparison therewith. As for the eco-related evaluation referred to here, a statistic, such as fuel with a high alcohol concentration being continuously supplied or the supply frequency of fuel with a high alcohol concentration increasing, for example, may calculated, as fuel that is supplied to the vehicle 200. This information may be provided to the user of the vehicle 200 or to the sales support server 100 periodically. This information may be used as information for reflecting on incentives or improving the eco-awareness of users, for example.

In step S506, the communication processing unit 204 transmits fuel information including the supply information stored in step S505 to the sales support server 100 via the communication unit 27. Note that the transmission referred to here may be performed every supply or a configuration may be adopted in which a predetermined number of pieces of fuel information are collectively transmitted. This processing flow is then ended.

### Incentive Provision Processing

The flow of processing for providing an incentive according to the result of evaluating the supplied fuel in the sales support server 100 will be described using FIG. 6. This processing is realized as a result of the incentive provision unit 106 shown in FIG. 3A being realized by a program held in the HDD 14 or the like being read out and executed by the CPU 11 of the sales support server 100.

In step S601, the incentive provision unit 106 acquires the fuel information transmitted from the vehicle 200. As described above using FIG. 5, fuel information is transmitted from the vehicle 200 side.

In step S602, the incentive provision unit 106 reads out the user information of the user corresponding to the fuel information from the user information DB 108.

In step S603, the incentive provision unit 106 reads out incentive conditions from the incentive conditions DB 110, based on the fuel information and corresponding user information.

In step S604, the incentive provision unit 106 determines the incentive to be provided to the corresponding user, based on the fuel information and incentive conditions.

In step S605, the incentive provision unit 106 holds the determined incentive in the user information DB 108 in association with the user information.

In step S606, the incentive provision unit 106 notifies the contents of the provided incentive to the vehicle 200. The notification method referred to here may, for example, involve display on the UI unit 26 of the vehicle 200, or may involve transmission to a mail address registered by the user as user information. This processing flow is then ended.

Note that, in step S505 of FIG. 5, the fuel evaluation unit 203 may perform processing for specifying a fuel product corresponding to the alcohol concentration estimated in step S504 from among the fuel products being sold in the store, based on the position at which fuel was supplied and the store information provided by the sales support server 100. The estimated alcohol concentration is corrected based on the alcohol concentration of fuel products disclosed in advance, thereby enabling the accuracy of the estimation result to be improved. Also, the fuel product supplied by the user can be specified based on the estimated alcohol concentration, and an incentive to purchase that fuel product can be reliably provided. Note that this specification may be performed on the sales support server 100 side rather than the vehicle side.

As described above, according to the present embodiment, when a user who uses a vehicle purchases a fuel product, it becomes possible to perform processing on the vehicle side, without purchase information thereof needing to be input or registered in a server. Incentives can then be provided to users who purchased fuel products, based on that information. Thus, it becomes possible to improve the convenience with which general users acquire and use incentives, with the aim of further expanding use of alternative fuels.

### Second Embodiment

The first embodiment described an example in which an alcohol sensor was used as means of the concentration detection unit 32. The present embodiment describes an example in which a knock sensor is used as means of the concentration detection unit 32.

### Knock Sensor

A knock sensor is able to detect knocking that occurs when fuel is combusting abnormally in an engine mounted in a vehicle. This abnormal combustion occurs due to the components of the fuel or a change of the traveling state, for example. On the vehicle side, in the case where the occurrence of knocking is detected by the knock sensor, the engine ignition timing is adjusted so as to suppress the occurrence of knocking. Specifically, in the case where the occurrence of knocking is detected, control (retardation) for delaying the timing from the ignition timing serving as a basis is performed. The retardation amount in this case is determined based on the value detected by the knock sensor.

In the present embodiment, the concentration of fuel is estimated, based on control information that is controlled, according to the change in output of the knock sensor. Note that since denoising on detected frequencies of vibration or sound and extraction of specific frequencies by the knock sensor in the concentration detection unit 32 use known methods, detailed description thereof here will be omitted. Accordingly, the knock sensor referred to here is assumed to perform processing such as acquisition of frequencies of vibration and the like that occur, extraction of specific frequency components for determining knocking, determination of knocking from extracted frequency components, and conversion to and output of voltage values corresponding to knocking that occurs. The drive unit 28 controls the ignition timing of the engine unit 29, based on the output voltages of the knock sensor.

### Processing Flow

### Fuel Evaluation Processing

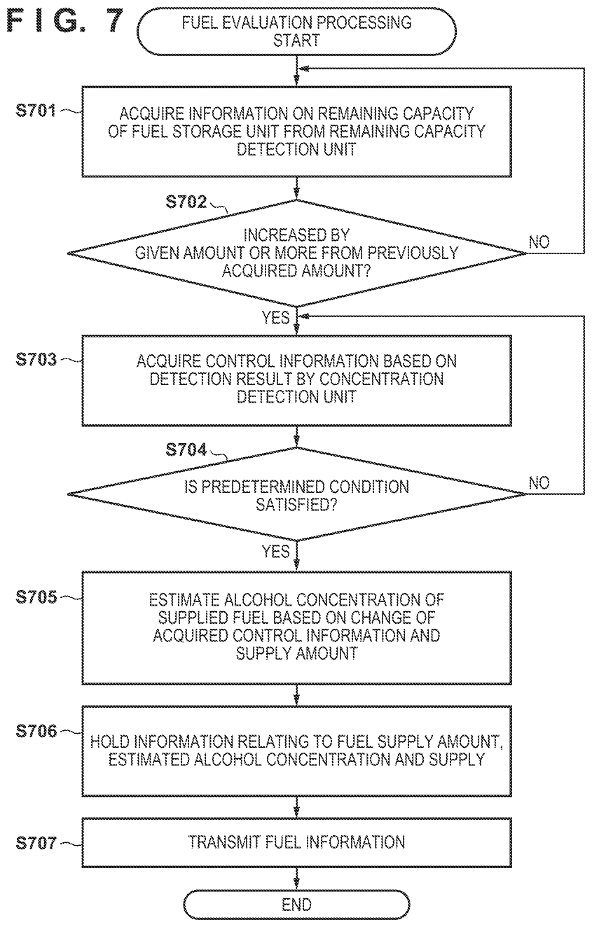

The flow of processing for evaluating supplied fuel in the vehicle 200 will be described using FIG. 7. This processing is executed instead of FIG. 5 described in the first embodiment. This processing is realized as a result the various processing units shown in FIG. 3B being realized by programs held in the HDD 25 or the like being read out and executed by the CPU 22 of the vehicle 200. This processing is started together with startup of the vehicle 200.

In step S701, the remaining capacity acquisition unit 201 acquires information on the remaining capacity of fuel that is stored in the fuel storage unit 30 from the remaining capacity detection unit 31. The information acquired here is stored in a storage unit such as the HDD 25 as history information according to changes therein.

In step S702, the remaining capacity acquisition unit 201 determines whether the remaining capacity has increased by a given amount or more as the amount of change from the previous remaining capacity, with reference to the history of acquired information on the remaining capacity. The given amount referred to here is assumed to be an amount defined in advance and held in the ROM 24 or the like. If it is determined that the amount of change is an increase greater than or equal to the given amount (YES in step S702), the processing advances to step S703 assuming that fuel has been newly supplied, and, if it is determined that the amount of change has decreased or has increased by less than the given amount (NO in step S702), the processing returns to step S701, and acquisition of information on the remaining capacity is continued.

In step S703, the concentration estimation unit 202 acquires control information that is based on the detection result from the concentration detection unit 32. As described above, in the present embodiment, a knock sensor is used as the concentration detection unit 32. Specifically, the concentration estimation unit 202 acquires the control amount (e.g., retardation amount) of the ignition timing of the engine that is performed by the vehicle 200, based on the detection result of the knock sensor. Note that information on the detection result (control amount) detected by the concentration detection unit 32 is stored in a storage unit such as the HDD 25 as history information according to changes therein.

In step S704, the concentration estimation unit 202 determines whether a predetermined condition is satisfied. The predetermined condition referred to here includes the case where the vehicle 200 travels a given distance or travels for a given time from when control information that is based on the detection result by the knock sensor starts being acquired in step S703 after a given amount of fuel is supplied. The given distance and given time referred to here are assumed to be defined in advance. During this traveled distance or traveled time, it is assumed that, on the vehicle 200 side, the ignition timing of the engine is adjusted, based on the detection results of the knock sensor, and the occurrence of knocking is gradually suppressed. In the meantime, control information continues to be acquired. The information used in determination referred to here is assumed to be acquired from the vehicle 200 side on a timely basis. If it is determined that the predetermined condition is not satisfied (NO in step S704), the processing returns to step S703, and the acquisition of control information that is based on the detection result of the knock sensor is continued. If it is determined that the predetermined condition is satisfied, the processing advances to step S705 (YES in step S704).

In step S705, the concentration estimation unit 202 estimates the alcohol concentration of the supplied fuel, based on the remaining capacity of fuel before supply, the supply amount of fuel, the alcohol concentration before supply, and the change in the control amount of the ignition timing that depends on the detection result of the knock sensor. The relative relationship between the alcohol concentration of fuel and the change in the control amount of ignition timing referred to here is defined in advance, and the concentration estimation unit 202 may estimate the alcohol concentration with reference to information on the change in the acquired control amount and information on this relative relationship.

In step S706, the fuel evaluation unit 203 stores the supplied amount of fuel, estimated alcohol concentration, supply date-time, supply place (position) and the like in association with each other in a storage unit such as the HDD 25 as information on the supply history. At this time, a configuration may be adopted in which the previous supply information is referred to, and eco-related evaluation is performed from the result of comparison therewith. As for the eco-related evaluation referred to here, similarly to the first embodiment, a statistic, such as fuel with a high alcohol concentration being continuously supplied or the supply frequency of fuel with a high alcohol concentration increasing, for example, may calculated, as fuel that is supplied to the vehicle 200. This information may be provided to the user of the vehicle 200 or to the sales support server 100 periodically. This information may be used as information for reflecting on incentives or improving the eco-awareness of users, for example.

In step S707, the communication processing unit 204 transmits fuel information including the supply information stored in step S706 to the sales support server 100 via the communication unit 27. Note that the transmission referred to here may be performed every supply or a configuration may be adopted in which a predetermined number of pieces of fuel information are collectively transmitted. This processing flow is then ended.

As described above, according to the present embodiment, when a user who uses a vehicle purchases a fuel product, it becomes possible to perform processing on the vehicle side, without purchase information thereof needing to be input or registered in a server. Incentives can then be provided to users who purchased fuel products, based on that information. Thus, it becomes possible to improve the convenience with which general users acquire and use incentives, with the aim of further expanding use of alternative fuels.

### Other Embodiments

Also, in the above example, vehicles communicate with a sales support server, but the present invention is not limited thereto. For example, the present invention may be applied to an in-vehicle device capable of communicating with the vehicle main body and a sales support server.

Also, information for prompting use of incentives provided as a result of the above processing may be presented. For example, if the provided incentive can be used at a store, store information relating to the store may be presented on a user terminal. Additionally, in the case where an incentive can be used at a place other than at stores where fuel products are purchased, notification for prompting guidance to the place and use of the incentive may be performed on the user terminal (e.g., application). For example, a contribution destination for contributions to environmental activity, a retail destination for incentives for supporting purchase of environmentally friendly products, and an access destination for environmental activity-related web content are given as examples.

### SUMMARY OF THE EMBODIMENTS

1. A fuel evaluation apparatus (e.g., 21) of the above embodiments is a fuel evaluation apparatus for evaluating fuel supplied to a vehicle (e.g., 200), characterized by comprising:

first acquisition means (e.g., 31, 201) for acquiring information on a remaining capacity of a fuel storage unit (e.g., 30) provided in the vehicle;

second acquisition means (e.g., 32, 202) for acquiring information on an alcohol concentration of fuel within the fuel storage unit;

calculation means (e.g., 201) for calculating a supply amount of fuel supplied to the fuel storage unit, based on the information on the remaining capacity acquired by the first acquisition means;

evaluation means (e.g., 203) for evaluating the alcohol concentration of the fuel supplied to the fuel storage unit, based on the supply amount of fuel calculated by the calculation means and a change in the alcohol concentration before and after supply of the fuel to the fuel storage unit that is based on the information on the alcohol concentration acquired by the second acquisition means; and

notification means (e.g., 204) for notifying the supply amount of fuel calculated by the calculation means and a result of the evaluation by the evaluation means.

According to this embodiment, when a user who uses a vehicle purchases a fuel product, it becomes possible to perform processing on the vehicle side, without purchase information thereof needing to be input or registered in a server. Thus, it becomes possible to improve the convenience with which general users acquire and use incentives, with the aim of the further expanding use of alternative fuels.

2. The fuel evaluation apparatus of the above embodiments, characterized in that the second acquisition means acquires information on an alcohol concentration measured using an ethanol sensor.

According to this embodiment, fuel concentration detection by an ethanol sensor becomes possible.

3. The fuel evaluation apparatus of the above embodiments, characterized in that the second acquisition means acquires information on the alcohol concentration, by acquiring at least one of information relating to a change in an output of a knock sensor and information relating to a change in an ignition timing of an engine of the vehicle, and estimating the alcohol concentration within the fuel storage unit using the acquired information.

According to this embodiment, fuel concentration detection by the knock sensor becomes possible.

4. The fuel evaluation apparatus of the above embodiments, characterized in that the second acquisition means acquires information on an alcohol concentration measured while predetermined travel is performed, after a detection value of the first acquisition means increases.

According to this embodiment, the detection system for fuel concentration detection can be improved.

5. The fuel evaluation apparatus of the above embodiments, characterized in that the predetermined travel is travel for a predetermined time or travel for a predetermined distance.

According to this embodiment, the detection system of fuel concentration detection can be improved.

6. The fuel evaluation apparatus of the above embodiments, characterized in that the fuel is fuel containing bioethanol as a component.

According to this embodiment, it becomes possible to promote use of bioethanol.

7. The fuel evaluation apparatus of the above embodiments, characterized in that the notification means further notifies position information of where the vehicle is located at a point in time at which a detection value of the first acquisition means increases.

According to this embodiment, the accuracy with which supplied fuel is recognized can be improved.

8. A control method for a fuel evaluation apparatus of the above embodiments is a control method for a fuel evaluation apparatus (e.g., 21) for evaluating fuel supplied to a vehicle (e.g., 200), the method characterized by comprising:

a first acquisition step of acquiring information on a remaining capacity of a fuel storage unit (e.g., 30) provided in the vehicle;

a second acquisition step of acquiring information on an alcohol concentration of fuel within the fuel storage unit;

a calculation step of calculating a supply amount of fuel supplied to the fuel storage unit, based on the information on the remaining capacity acquired in the first acquisition step;

an evaluation step of evaluating the alcohol concentration of the fuel supplied to the fuel storage unit, based on the supply amount of fuel calculated in the calculation step and a change in the alcohol concentration before and after supply of the fuel to the fuel storage unit that is based on the information on the alcohol concentration acquired in the second acquisition step; and

a notification step of notifying the supply amount of fuel calculated in the calculation step and a result of the evaluation in the evaluation step.

According to this embodiment, when a user who uses a vehicle purchases a fuel product, it becomes possible to perform processing on the vehicle side, without purchase information thereof needing to be input or registered in a server. Thus, it becomes possible to improve the convenience with which general users acquire and use incentives, with the aim of the further expanding use of alternative fuels.

9. A program of the above embodiments is a program for causing a computer (e.g., 22) to function as:

first acquisition means (e.g., 31, 201) for acquiring information on a remaining capacity of a fuel storage unit (e.g., 30) provided in a vehicle;

second acquisition means (e.g., 32, 202) for acquiring information on an alcohol concentration of fuel within the fuel storage unit;

calculation means (e.g., 201) for calculating a supply amount of fuel supplied to the fuel storage unit, based on the information on the remaining capacity acquired by the first acquisition means;

evaluation means (e.g., 203) for evaluating the alcohol concentration of the fuel supplied to the fuel storage unit, based on the supply amount of fuel calculated by the calculation means and a change in the alcohol concentration before and after supply of the fuel to the fuel storage unit that is based on the information on the alcohol concentration acquired by the second acquisition means; and

notification means (e.g., 27, 204) for notifying the supply amount of fuel calculated by the calculation means and a result of the evaluation by the evaluation means.

According to this embodiment, when a user who uses a vehicle purchases a fuel product, it becomes possible to perform processing on the vehicle side, without purchase information thereof needing to be input or registered in a server. Thus, it becomes possible to improve the convenience with which general users acquire and use incentives, with the aim of the further expanding use of alternative fuels.

1. A fuel evaluation apparatus for evaluating fuel supplied to a vehicle, the fuel evaluation apparatus performing a method comprising:
acquiring information on a remaining capacity of a fuel storage unit provided in the vehicle;
acquiring information on an alcohol concentration of fuel within the fuel storage unit;
calculating a supply amount of fuel supplied to the fuel storage unit, based on the acquired information on the remaining capacity;
evaluating the alcohol concentration of the fuel supplied to the fuel storage unit, based on the calculated supply amount of fuel and a change in the alcohol concentration before and after supply of the fuel to the fuel storage unit that is based on the acquired information on the alcohol concentration; and
transmitting fuel information comprising the calculated supply amount of fuel and a result of the evaluation to external apparatus determining incentives according to the fuel information.

2. The fuel evaluation apparatus according to claim 1, wherein the acquiring information on an alcohol concentration of fuel includes acquiring information on an alcohol concentration measured using an ethanol sensor.

3. The fuel evaluation apparatus according to claim 1, wherein the acquiring information on an alcohol concentration of fuel includes acquiring information on the alcohol concentration, by acquiring at least one of information relating to a change in an output of a knock sensor and information relating to a change in an ignition timing of an engine of the vehicle, and estimating the alcohol concentration within the fuel storage unit using the acquired information.

4. The fuel evaluation apparatus according to claim 1, wherein the acquiring information on an alcohol concentration of fuel includes acquiring information on an alcohol concentration measured while predetermined travel is performed, after a detection value of the acquired remaining capacity of the fuel storage unit increases.

5. The fuel evaluation apparatus according to claim 4, wherein the predetermined travel is travel for a predetermined time or travel for a predetermined distance.

6. The fuel evaluation apparatus according to claim 1, wherein the fuel is fuel containing bioethanol as a component.

7. The fuel evaluation apparatus according to claim 1, wherein the transmitting fuel information includes, to the external apparatus, position information of where the vehicle is located at a point in time at which a detection value of the acquired remaining capacity of the fuel storage unit increases.

8. A control method for a fuel evaluation apparatus for evaluating fuel supplied to a vehicle, the method comprising:
acquiring information on a remaining capacity of a fuel storage unit provided in the vehicle;
acquiring information on an alcohol concentration of fuel within the fuel storage unit;
calculating a supply amount of fuel supplied to the fuel storage unit, based on the acquired information on the remaining capacity;
evaluating the alcohol concentration of the fuel supplied to the fuel storage unit, based on the calculated supply amount of fuel and a change in the alcohol concentration before and after supply of the fuel to the fuel storage unit that is based on the acquired information on the alcohol concentration; and
transmitting fuel information comprising the calculated supply amount of fuel and a result of the evaluation to external apparatus determining incentives according to the fuel information.

9. A computer-readable storage medium storing a program for causing a computer to perform:
acquiring information on a remaining capacity of a fuel storage unit provided in a vehicle;
acquiring information on an alcohol concentration of fuel within the fuel storage unit;
calculating a supply amount of fuel supplied to the fuel storage unit, based on the acquired information on the remaining capacity;
evaluating the alcohol concentration of the fuel supplied to the fuel storage unit, based on the calculated supply amount of fuel and a change in the alcohol concentration before and after supply of the fuel to the fuel storage unit that is based on the acquired information on the alcohol concentration; and
communicating fuel information comprising the calculated supply amount of fuel and a result of the evaluation to external apparatus determining incentives according to the fuel information.
